(19) United States
(12) Patent Application Publication
(10) Pub. No.: US 2024/0160275 A1
(43) Pub. Date: May 16, 2024

(54) METHOD OF IMPLEMENTING CONTENT REACTING TO USER RESPONSIVENESS IN METAVERSE ENVIRONMENT

(71) Applicant: ELECTRONICS AND TELECOMMUNICATIONS RESEARCH INSTITUTE, Daejeon (KR)

(72) Inventors: Ki-Hong KIM, Daejeon (KR); Yong Wan KIM, Daejeon (KR); Jin Sung CHOI, Daejeon (KR)

(21) Appl. No.: 18/212,875

(22) Filed: Jun. 22, 2023

(30) Foreign Application Priority Data
Nov. 16, 2022 (KR) 10-2022-0153756

(51) Int. Cl. G06F 3/01; G06T 11/60; G06V 10/82; G06V 40/70

(52) U.S. Cl. CPC G06F 3/011; G06T 11/60; G06V 10/82; G06V 40/70; G06F 2203/011

(57) ABSTRACT

Provided is a method of providing content in a metaverse environment, the method including: providing, by a content providing apparatus, content to a user of a metaverse; acquiring, by the content providing apparatus, user responsiveness information of the user corresponding to the content; acquiring, by the content providing apparatus, a user responsiveness based on the user responsiveness information using a multimodal artificial intelligence model; and providing, by the content providing apparatus, modified content based on the user responsiveness to the metaverse environment.

### CROSS-REFERENCE TO RELATED APPLICATION

This application claims priority to Korean Patent Applications No. 10-2022-00153756, filed on Nov. 16, 2022, which is hereby incorporated by reference in its entirety.

### BACKGROUND

### 1. Field of the Invention

The present invention relates to a method of changing content provided in a metaverse environment based on the responsiveness of a plurality of users who access the metaverse environment.

### 2. Description of Related Art

In recent years, various companies have been producing various media or content targeting the general public for advertising or promoting corporate events, and providing websites, mass media, and the like. In addition, with the rapid rise of non-face-to-face services, metaverse-type content that allows multiple users to interact online is being activated.

However, since the content provided in the metaverse environment is produced based on the author's experience and creativity, there is a lack of a methodology for quantitatively checking the level of satisfaction and favorability of users with the content.

Recently, there have been examples in which emotional states of users are identified by sensing biometric signals or actions to use the identified emotional state as a basis for analyzing service effectiveness.

Specifically, the examples include a technology of analyzing a facial image of a user watching content to analyze a user's emotional state, a technology of analyzing an electroencephalogram (EEG) detected in the frontal lobe with a biometric signal sensor to derive a user's emotion.

However, the conventional methods have focused on identifying emotional states of people who encounter or experience content or have been used to evaluate the effectiveness of content based on the analyzed emotional states, and may lack a methodology for changing content provided in a metaverse environment in real time.

### SUMMARY OF THE INVENTION

The present disclosure is provided to, using artificial intelligence, change content provided to a plurality of users accessing a metaverse environment based on a user responsiveness.

The present disclosure discloses a method of providing content in a metaverse environment, the method including: providing, by a content providing apparatus, content to a user of a metaverse; acquiring, by the content providing apparatus, user responsiveness information of the user corresponding to the content; acquiring, by the content providing apparatus, a user responsiveness based on the user responsiveness information using a multimodal artificial intelligence model; and providing, by the content providing apparatus, modified content based on the user responsiveness to the metaverse environment.

Also, the user responsiveness information may include at least one type information among facial expression information, gaze information, and motion information of the user, wherein the acquiring, by the content providing apparatus, of the user responsiveness based on the user responsiveness information using the multimodal artificial intelligence model may include: determining whether the content is at a predetermined event time point; and inputting the at least one type of information among the facial expression information, the gaze information, and the motion information of the user into the multimodal artificial intelligence model to acquire the user responsiveness.

Also, the providing, by the content providing apparatus, of the modified content based on the user responsiveness to the metaverse environment may include loading an Effect asset and a prop from a resource database to output the modified content to the metaverse environment.

Also, the acquiring, by the content providing apparatus, of the user responsiveness based on the user responsiveness information using the multimodal artificial intelligence model may include determining whether the content is at a predetermined content start time point; and inputting the at least one type of the facial expression information, the gaze information, and the motion information of the user into the multimodal artificial intelligence model to acquire the user responsiveness.

Also, the providing, by the content providing apparatus, of the modified content based on the user responsiveness to the metaverse environment may include changing a scenario to correspond to a level of the acquired user responsiveness among scenarios of the content and outputting the changed scenario, or outputting and providing the changed scenario after a currently ongoing scenario.

Also, the multimodal artificial intelligence model may include a plurality of convolutional neural networks, and each of the plurality of convolutional neural networks may output a feature corresponding to each of facial expression information, gaze information, and motion information of the user.

Also, the multimodal artificial intelligence model may include a recurrent neural network, and the recurrent neural network may output a user responsiveness based on the facial expression information, the gaze information, and the motion information of the user which may be output from each of the plurality of convolutional neural networks.

An apparatus for providing content in a metaverse environment according to an embodiment of the present invention includes: a reproducing unit configured to provide content to a user of a metaverse; a photographing unit configured to acquire user responsiveness information of the user corresponding to the content; and a processor configured to acquire a user responsiveness based on the user responsiveness information using a multimodal artificial intelligence model and provide modified content based on the user responsiveness to the metaverse environment. Also, when the content is at a predetermined event time point, the processor may input the at least one type of information among the facial expression information, the gaze information, and the motion information of the user into the multimodal artificial intelligence model to acquire the user responsiveness.

Also, when the content is at the predetermined event time point, the processor may load an Effect asset and a prop from a resource database to output the modified content to the metaverse environment.

Also, when the content is at a predetermined content start time point, the processor may input the at least one type of information among the facial expression information, the gaze information, and the motion information of the user into the multimodal artificial intelligence model to acquire the user responsiveness.

Also, the processor may change a scenario to correspond to a level of the acquired user responsiveness among scenarios of the content and output the changed scenario, or output and provide the changed scenario after a currently ongoing scenario.

A training apparatus for providing content to a metaverse environment using an artificial intelligence according to an embodiment of the present invention includes: a stimulus storage unit configured to store a stimulus moving image, a stimulus still image, or stimulus sound designed to induce an emotion related to a user responsiveness; a reproducing unit configured to reproduce the content; a photographing unit configured to acquire facial expression information, gaze information, posture information, and motion information of a user using at least one camera; a user input processing unit configured to store specific sections set by the user; and a labeling unit configured to provide content of the specific section to the user, acquire a responsiveness score based on a predetermined criterion, and label pieces of image sequence data of the specific section with the acquired responsiveness scores to generate training data.

Also, in response to a preceding time point being set as an onset point and a following time point being set as an ending point among time points marked by the user clicking a mouse or a remote control, the specific section may include a part between the onset point and the ending point.

### BRIEF DESCRIPTION OF THE DRAWINGS

FIG. 1 illustrates an example of a content providing environment according to an embodiment of the present invention.

FIG. 2 is a view illustrating a process of training an artificial intelligence model according to an embodiment of the present invention.

FIG. 3 relates to a process of a user marking a part having a high responsiveness during a training process of an artificial intelligence model according to an embodiment of the present invention.

FIG. 4 is a view illustrating a configuration of an artificial intelligence model according to an embodiment of the present invention.

FIG. 5 is a view illustrating a process of providing content based on a user responsiveness according to an embodiment of the present invention.

FIG. 6 is a view illustrating an example of a process of providing content based on a user responsiveness according to an embodiment of the present invention.

### DETAILED DESCRIPTION OF EXEMPLARY EMBODIMENTS

In the following detailed description, since the technology to be described below can have various changes and various embodiments, specific embodiments will be illustrated in the accompanying drawings and described in detail. However, this is not intended to limit the technology described below to specific embodiments, and it should be understood to include all modifications, equivalents, or substitutes included in the spirit and scope of the technology described below.

Terms such as first, second, A, B, etc. may be used to describe various elements, but the elements are not limited by the above terms, and are merely used to distinguish one element from another. For example, without departing from the scope of the technology described below, a first element may be referred to as a second element, and similarly, the second element may be referred to as the first element. The term “and/or” includes any combination of a plurality of related recited items or any of a plurality of related recited items.

In terms used in this specification, singular expressions should be understood to include plural expressions unless clearly interpreted differently in the context, and terms such as “comprising” refer to the described features, numbers, steps, operations, and components, parts or combinations thereof, but it should be understood that it does not exclude the possibility of the presence or addition of one or more other features, numbers, steps, operations, and components, parts or combinations thereof.

Prior to a detailed description of the drawings, it is to be clarified that the classification of components in the present specification is merely a classification for each component responsible for each main function. That is, two or more components to be described below may be combined into one component, or one component may be divided into two or more for each subdivided function. In addition, each component to be described below may additionally perform some or all of the functions of other components in addition to its main function, and some of the main functions of each component may be performed by other components. Of course, some of the main functions of each component may be exclusively performed by other components.

In addition, in performing a method or method of operation, each process constituting the method may occur in a different order from the specified order unless a specific order is clearly described in the context. That is, each process may occur in the same order as specified, may be performed substantially simultaneously, or may be performed in the reverse order.

First, definitions of terms used in the following description are as follows.

The term “Metaverse” according to an embodiment of the present disclosure may be a virtual world in which real life is connected to legally recognized activities, such as work, finance, and learning. Specifically, as a higher concept of virtual reality and augmented reality, a metaverse may be a system that expands reality into a digital-based virtual world to enable all activities in a virtual space.

The term “Content” according to an embodiment of the present disclosure may include video, document, image, virtual reality (VR), and augmented reality (AR) content, and may include information that is decodable through an electronic device or application.

The term “User responsiveness information” according to an embodiment of the present disclosure may include various types of information for deriving a “user responsiveness” to be described below. For example, the user responsiveness information may include facial expression information, gaze information, motion information (a posture, a motion, a gesture, etc.) of a user.

The term “User responsiveness” may be a score representing a degree according to the user responsiveness information indicated as a score. For example, when the degree of a user's reaction exceeds a preset degree based on a feature extracted from user responsiveness information, the user responsiveness may be measured as high, and when the degree of the user's reaction is lower than the preset degree, the user responsiveness may be measured as low.

A multimodal artificial intelligence model according to an embodiment of the present disclosure may refer to an artificial network model designed to derive a user responsiveness based on user responsiveness information.

The term “metaverse service provider” according to an embodiment of the present disclosure may provide a metaverse environment, and a metaverse service may be implemented through a specific terminal, server, or cloud.

In addition, a “content provider” may refer to an author or provider of content executed in the metaverse environment. A terminal or server used by the content provider may be named an apparatus for providing content (hereinafter referred to as a content providing apparatus).

Depending on implementation examples, the metaverse service provider and the content provider may be the same.

A user according to an embodiment of the present disclosure may correspond to a user accessing the metaverse environment, and a user terminal may refer to an electronic device used by the user. Examples of user terminals may include various devices capable of accessing a metaverse, such as a mobile phone, a laptop computer, a head mounted display (HMD), a VR device, and the like.

In the following description, an action performed by a user in a metaverse environment may be transmitted to a service provider or a content providing apparatus through a user terminal, and a user terminal, a content provider terminal, and a service provider terminal may include devices for processing input data uniformly and performing calculations required to provide content according to a specific model or algorithm. For example, the content providing apparatus may be implemented in the form of a personal computer (PC), a server on a network, a smart device, a chipset in which a design program is embedded, and the like.

Hereinafter, a configuration for changing content based on a responsiveness to content expressed by a plurality of users participating in a virtual content environment in a metaverse environment will be described.

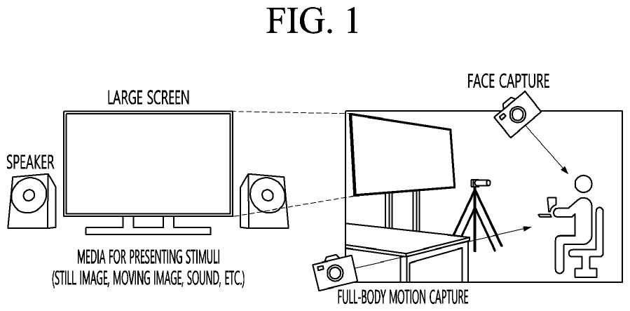

FIG. 1 illustrates an example of a content providing environment according to an embodiment of the present invention.

Meanwhile, in FIG. 1, a content providing environment for acquiring a user responsiveness to content is illustrated, and a system for implementing the content providing environment may be implemented similar to an artificial intelligence model training environment.

The system may include an output device including a display and a speaker, at least one photographing unit 10 for receiving user motion information, and a user terminal for providing a metaverse environment. Depending on implementation examples, an output unit and a camera mounted in a user terminal may serve as the output device and the camera.

From the perspective of a content provider, the content providing system shown in FIG. 1 may be a system for training an artificial intelligence model.

Specifically, the content providing system may be a system that collects training data required for training an artificial intelligence model to understand how users react or respond to content provided by a content provider.

When collecting training data, in a situation in which users are watching specific content, data (a still image or a moving image) on a facial expression, a gaze, and a body motion, of a user may be acquired through the photographing unit (including a camera and the like) provided in the user terminal or the system.

Meanwhile, it is important to acquire various pieces of training data to improve the performance of the artificial intelligence model. For example, it is very important to acquire images of various motions, gazes, and facial expressions regarding a reaction shown by a user.

The training data of the artificial intelligence model may include various emotional states related to being positive and negative, such as joy, touching, and sadness, and stimulus information capable of inducing the emotional states, such as still images, moving images, and sounds.

The stimulus information may be acquired by photographing the way in which each user reacts to content provided to the user through a display or speaker using a photographing unit. The acquired data may be databased as training data.

Training data according to an embodiment of the present invention may be formed based on various factors, such as the preference, stimulus threshold, race, sex, age, nationality, and language of a user.

For example, even with stimulus information of the same content image or sound, a plurality of users may each have a different responsiveness based on various factors, such as preference, race, sex, age, nationality, language, and the like, and thus in a preparation operation before photography for generating training data, each user may be allowed to freely select stimulus information he or she prefers in association with a specific emotional state so that training data may be generated.

Hereinafter, a process of training an artificial intelligence model according to the present invention will be described.

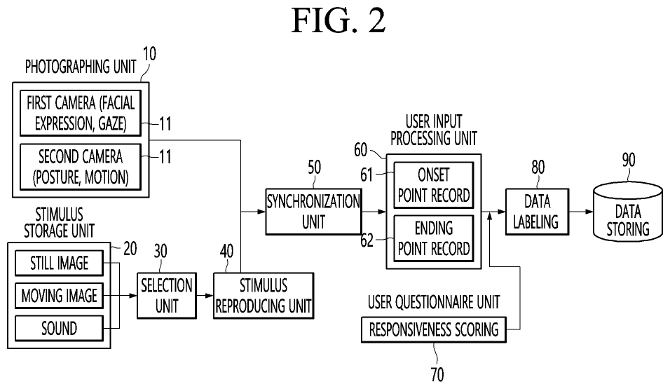

FIG. 2 is a view illustrating a process of training an artificial intelligence model according to an embodiment of the present invention.

Referring to FIG. 2, a process in which at least one piece of content is provided to a user to acquire a user responsiveness corresponding thereto, and the user responsiveness is converted and stored in a database to generate training data is illustrated.

The subject of the operations shown in FIG. 2 may be a training apparatus, and the training apparatus may be provided as a separate device to store a training database, and the stored database may be provided to a content providing apparatus or transmitted to a server for training. In addition, the training apparatus may be a content providing apparatus.

After training of an artificial intelligence model is performed by the training apparatus, a content providing apparatus may acquire the user responsiveness in the same method as the training apparatus has performed the operation. Therefore, the content providing apparatus may be understood as including all components of the training apparatus.

According to an embodiment of the present disclosure, the training apparatus may include a stimulus storage unit 20 that stores content, a selection unit 30 that selects content, a stimulus reproducing unit 40 that reproduces content, a photographing unit 10 that photographs a user, a synchronization unit 50 that synchronizes the photographing unit 10 and the stimulus reproducing unit 40, a user input processing unit 60 that processes user input, a user questionnaire unit 70 that stores user questionnaires, and storages 80 and 90 that perform data labeling and storage. The components may be electrically or structurally connected to each other and may be operated by at least one processor.

The selection unit 30 may select content from the stimulus storage unit 20 in which a stimulus video, a stimulus still image, or stimulus sound designed to induce an emotion related to a responsiveness, and the stimulus reproducing unit 40 may reproduce the selected content.

In this case, the stimulus reproducing unit 40 may be synchronized with the photographing unit 10, and the photographing unit 10 may acquire facial expression information, gaze information, posture information, and motion information of a user using at least one camera.

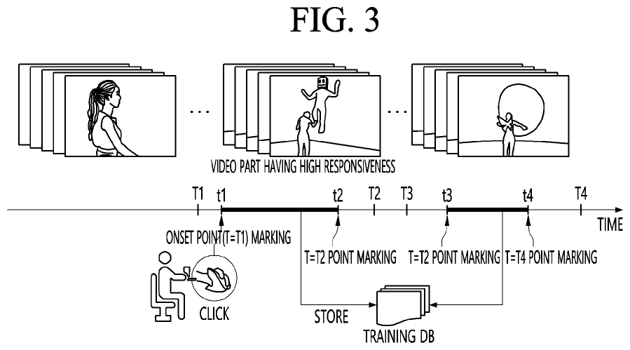

The user input processing unit 60 may store specific sections set by a user watching content. In this case, the specific section may be marked by clicking a user's mouse or remote control, and among the time points of markings, a preceding time point may be set as an onset point 61 and a following time point may be set as an ending point 62, and a part between the onset point 61 and the ending point 62 may be set as a specific section. This will be described in detail in FIG. 3 below.

The user questionnaire unit 70 may provide content of the specific section to the user again and acquire an appropriate responsiveness score based on a predetermined criterion through a questionnaire.

The labeling unit 80 and the data storage unit 90 may label pieces of video sequence data of the specific section with responsiveness scores determined by the user, thereby generating training data.

FIG. 3 relates to a process of a user marking a part with a high responsiveness during a training process of an artificial intelligence model according to an embodiment of the present invention.

According to an embodiment of the present invention, training data may be generated as a responsiveness score according to facial expression, gaze information, posture information, and motion information acquired in a specific section among all sections of content. In other words, “facial expression information, gaze information, posture information, and motion information of a user” and “user responsiveness” (a score) of a specific section may be stored as a training data pair.

On the other hand, at least one type of information among facial expression information, gaze information, posture information, and motion information of a user may be used.

The specific section may be set by a user. For example, the training apparatus may request the user to click (onset point) a wireless mouse or remote control at a point in time when a scene section with high preference appears while watching video content, and click once more (ending point) at a point in which when it is determined that the preference has decreased, thereby setting a specific section of the content.

In response to the specific section being set, the photographing unit 10 may photograph a facial expression, a gaze, a posture, and a motion of the user during the specific section to acquire facial expression information, gaze information, posture information, and motion information of the user. In addition, the responsiveness score of the specific section may be acquired by the user input at a later time. On the other hand, the specific section may be set in the content in advance by the content provider, and when a section set by the content provider and a specific section set by the user are different in a process of generating training data in practice, the facial expression data, gaze data, posture data, and motion data of the user acquired from the specific section set by the user may be used for training.

With such a configuration, since users are continuously photographed with a camera for a long time, a part showing a high responsiveness may be retrieved, and only an image of a user's facial expression and motion corresponding to when the user was watching the retrieved part may be acquired as valid data for training.

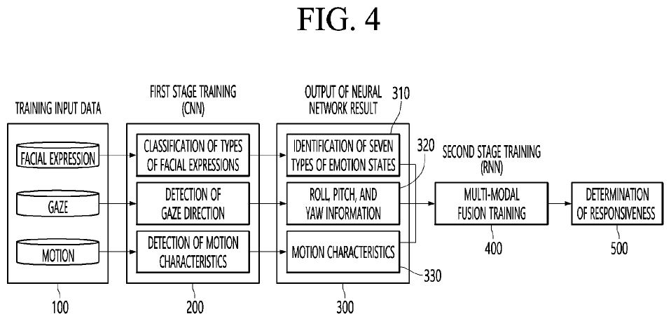

FIG. 4 is a view illustrating a configuration of an artificial intelligence model according to an embodiment of the present invention.

Referring to FIG. 4, the artificial intelligence model may include a convolutional neural network (CNN) 200 and a recurrent neural network (RNN) 400. Specifically, input data 100 for training may input to the CNN 200, and a result value 300 output from the CNN 200 may be input to the RNN 400, so that a final result 500 may be derived.

As described above, the input data 100 required for artificial intelligence learning may include various pieces of facial expression information, gaze information, posture information, and motion information acquired from each of a plurality of users. Such information as facial expression information, gaze information, posture information, and motion information is highly related to an emotional state of the user, such as content immersion, satisfaction, responsiveness, and the like. However, the form of reaction externally expressed or shown by each user may be different between people.

For example, the form of reaction may be different based on race, age, personality, culture, reaction threshold, etc. In the case of a specific person, the degree of responsiveness may be revealed in the facial expression, while in the case of another person, the degree of responsiveness may be easily identified by a large motion and the like.

Therefore, there is a need to objectively grasp the degree of response of people who encounter or experience content services by simultaneously acquiring several characteristic external reaction aspects, such as a facial expression, a gaze, and a body posture taken by users. This wad described with reference to FIG. 2.

The artificial intelligence model according to an embodiment of the present invention may input facial expression information, gaze information, and motion information (which may include posture information) of a user to the CNN 200, to detect a feature of each piece of the information.

For example, the CNN 200 may include a convolution neural network, and the CNN model may include three CNNs respectively corresponding to facial expression information, gaze information, and motion information.

Specifically, the CNN 200 may classify the type of facial expression from the facial expression information, detect a gaze direction from the gaze information, and detect a motion characteristic from the motion information.

For example, the CNN 200 may classify a plurality of emotional situations through facial expressions expressed as reactions to the content. Examples of the emotions may include emotions, such as Neutral, Happy, Sad, Surprised, Angry, Disgust, and Fear (310).

In addition, the CNN 200 may detect roll information, pitch information, and yaw information through a gaze direction according to a head posture to determine the degree of concentration on the content being reproduced.

In addition, the CNN 200 may detect whole body posture and motion or behavioral characteristics to determine the motion characteristics of the user (330). As described above, the artificial intelligence model may be a multi-modal model reflecting a plurality of pieces of characteristic information by detecting facial expression, gaze, and motion characteristics.

According to an embodiment of the present disclosure, the output result 300 of the individual CNNs classified and identified as described above may be used as an input parameter to an RNN.

Specifically, the RNN is a sequence model that processes inputs and outputs in units of sequences, and may be a multimodal model that considers all of facial expression information, gaze information, motion information, and posture information, which change according to a specific section.

According to an embodiment of the present disclosure, the RNN may output the degree of responsiveness based on facial expression information, gaze information, motion information, and posture information, which change according to a specific section, as a final result.

Meanwhile, in order to implement the CNN and the RNN according to the above embodiment, as the first stage training, the input data 100 for training, which is shown as a reaction to content, may be labeled on the result data 300 to perform training, and as the second stage training, a fusion training based on multimodal features that considers a facial expression, a gaze, a motion, and a behavior together may be used to finally evaluate the degree of responsiveness based on a ten point-score, and thus training may be performed in advance.

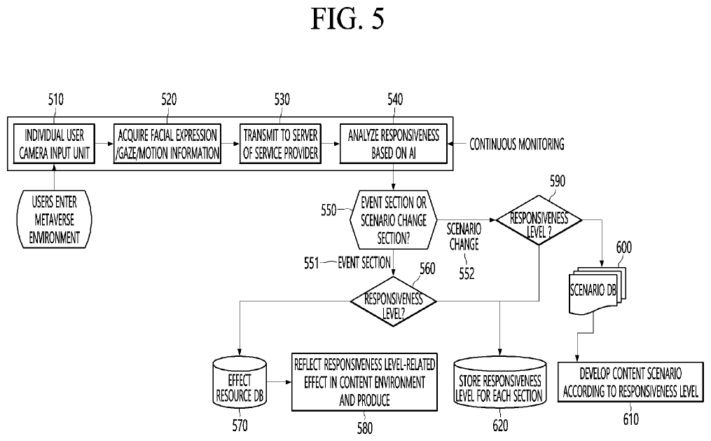

FIG. 5 is a view illustrating a process of providing content based on a user responsiveness according to an embodiment of the present invention.

First, the artificial intelligence model according to an embodiment of the present invention may be a type of algorithm, and may be internalized in the form of an engine on a metaverse content platform used by a content provider and provided. Through the provided artificial intelligence algorithm, users may watch various types of metaverse content provided by the content provider, and the content provider may identify a user responsiveness to content and modify or change the scenario of the provided content based on the user responsiveness.

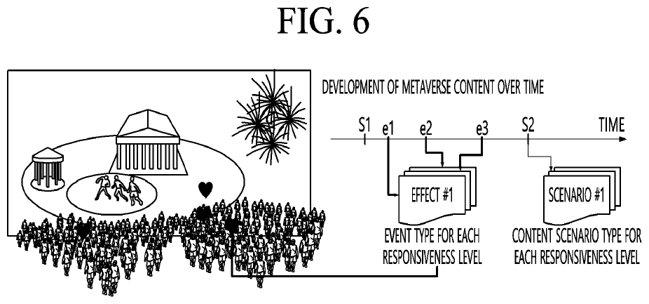

To this end, metaverse service providers may generate content and resources, such as Effect assets, for various scenarios according to the degree of responsiveness in advance and allow the firmware to be downloaded and stored on a server managed by the content provider or on each user's device. Hereinafter, an embodiment in which a content scenario is modified or changed will be described in detail with reference to FIGS. 5 and 6.

FIG. 6 is a view illustrating a metaverse content scenario change according to an embodiment of the present invention.

Referring to FIG. 6, upon entry into a predefined event e1, e2, or e3 in a situation in which metaverse content transmitted from a server of a service provider is developed over time, special effect events, such as setting off a firecracker, presenting a heart emoticon, other emotion emoticons, and emojis may be produced.

Alternatively, a content scenario may be modified or changed using the responsiveness acquired from a plurality of users who watch metaverse content at a content start point S1 or S2.

According to an embodiment of the present invention, a plurality of users may access a website or platform of a metaverse service provider using a user terminal and freely watch metaverse content. In this case, the content providing apparatus may provide metaverse content to the website or the metaverse platform using the server or the content provider terminal, thereby outputting the corresponding content to the user terminal.

A camera 510, such as a webcam, attached to each user's device may acquire the facial expression information, gaze information, and motion information of a user (520) and transmit the information to the content providing apparatus (530).

The artificial intelligence algorithm internalized in the content providing apparatus may analyze the responsiveness expressed by the corresponding user (540).

The content providing apparatus according to an embodiment of the present invention may, upon entry into an event or a content starting point, input facial expression information, gaze information, and motion information acquired from each user into the artificial intelligence model to derive the responsiveness identified from each of a plurality of users, and measure the overall responsiveness of all the plurality of users to the content being provided at the corresponding point in time or section using the derived responsiveness (560).

In this case, the overall responsiveness of all the plurality of users may be the sum or average value of a plurality of user responsivenesses.

Meanwhile, the responsiveness may be calculated by inputting at least one type of information among facial expression information, gaze information, and motion information acquired from the user into the artificial intelligence model.

The content providing apparatus according to an embodiment of the present disclosure may load various Effect assets and props from the resource database based on the responsiveness calculated at the point in time of an event e1, e2, or e3 (570) and reflect the Effect assets and props in an environment of the metaverse content, thereby providing modified metaverse content (580).

With such a configuration, users may be induced to have a greater interest in or continuously immerse themselves in the content being serviced.

Alternatively, the content providing apparatus may change a scenario that matches the level of responsiveness among a plurality of preset scenarios based on a content start point S1 or S2 and load the scenario (600) or may seamlessly connect the scenario to follow a currently ongoing scenario for developing (610), thereby providing modified or changed metaverse content.

With such a configuration, interactive content in which a plurality of users may determine a change direction of content may be provided.

With such a configuration, in a situation in which metaverse type content having a large number of scenarios is serviced online, reaction aspects expressed by a large number of users participating in an environment of the corresponding service or the overall responsiveness may be continuously analyzed by the engine in the content platform in real time, and accordingly the flow of content or the virtual environment of the metaverse content may be changed, thereby providing users with a new content experience and high satisfaction. Furthermore, service producers may be provided with feedback on the responsiveness of a large number of users such that the service producers may improve the quality of the service in operation or may plan or produce content for a new metaverse type service in the future using the feedback.

The present disclosure is implemented to, using artificial intelligence, change content provided by a content provider based on a user responsiveness to content provided to a plurality of users accessing a metaverse environment, thereby providing users of the metaverse environment with new content experiences and high satisfaction.

In addition, the present invention is implemented to provide service providers with objective result data on the responsiveness of users as feedback, thereby improving the quality of service.

Those skilled in the art should appreciate that a content providing apparatus, a user terminal, a terminal, and the like may be embodied by various illustrative logical blocks, modules, processors, means, circuits, and algorithm steps described in connection with the embodiments disclosed herein that may be implemented as electronic hardware, various types of program or design code (for the sake of convenience, referred to as software here), or combinations thereof.

The present invention described above may be embodied as computer-readable code on a medium in which a program is recorded. The computer-readable recording medium is any data storage device that can store data that can thereafter be read by a computer system. Examples of the computer-readable recording medium may include a hard disk drive (HDD), a solid-state drive (SSD), a silicon disk drive (SDD), a read-only memory (ROM), a random-access memory (RAM), a compact disc read only memory (CD-ROM), a magnetic tape, a floppy disk, an optical data storage, and the like.

What is claimed is:

1. A method of providing content in a metaverse environment, the method comprising:
providing, by a content providing apparatus, content to a user of a metaverse;
acquiring, by the content providing apparatus, user responsiveness information of the user corresponding to the content;
acquiring, by the content providing apparatus, a user responsiveness based on the user responsiveness information using a multimodal artificial intelligence model; and
providing, by the content providing apparatus, modified content based on the user responsiveness to the metaverse environment.

2. The method of claim 1, wherein the user responsiveness information includes at least one type information among facial expression information, gaze information, and motion information of the user,
wherein the acquiring, by the content providing apparatus, of the user responsiveness based on the user responsiveness information using the multimodal artificial intelligence model includes:
determining whether the content is at a predetermined event time point; and
inputting the at least one type of information among the facial expression information, the gaze information, and the motion information of the user into the multimodal artificial intelligence model to acquire the user responsiveness.

3. The method of claim 2, wherein the providing, by the content providing apparatus, of the modified content based on the user responsiveness to the metaverse environment includes loading an Effect asset and a prop from a resource database to output the modified content to the metaverse environment.

4. The method of claim 1, wherein the user responsiveness information includes at least one type of information among facial expression information, gaze information, and motion information of the user,
wherein, the acquiring, by the content providing apparatus, of the user responsiveness based on the user responsiveness information using the multimodal artificial intelligence model includes:
determining whether the content is at a predetermined content start time point; and
inputting the at least one type of the facial expression information, the gaze information, and the motion information of the user into the multimodal artificial intelligence model to acquire the user responsiveness.

5. The method of claim 4, wherein the providing, by the content providing apparatus, of the modified content based on the user responsiveness to the metaverse environment includes changing a scenario to correspond to a level of the acquired user responsiveness among scenarios of the content and outputting the changed scenario, or outputting and providing the changed scenario after a currently ongoing scenario.

6. The method of claim 1, wherein the multimodal artificial intelligence model includes a plurality of convolutional neural networks, and each of the plurality of convolutional neural networks outputs a feature corresponding to each of facial expression information, gaze information, and motion information of the user.

7. The method of claim 6, wherein the multimodal artificial intelligence model includes a recurrent neural network, and the recurrent neural network outputs a user responsiveness based on the facial expression information, the gaze information, and the motion information of the user which are output from each of the plurality of convolutional neural networks.

8. An apparatus for providing content in a metaverse environment, the apparatus comprising:
a reproducing unit configured to provide content to a user of a metaverse;
a photographing unit configured to acquire user responsiveness information of the user corresponding to the content; and
a processor configured to acquire a user responsiveness based on the user responsiveness information using a multimodal artificial intelligence model and provide modified content based on the user responsiveness to the metaverse environment.

9. The apparatus of claim 8, wherein the user responsiveness information includes at least one type of information among facial expression information, gaze information, and motion information of the user, and
when the content is at a predetermined event time point, the processor inputs the at least one type of information among the facial expression information, the gaze information, and the motion information of the user into the multimodal artificial intelligence model to acquire the user responsiveness.

10. The apparatus of claim 9, wherein, when the content is at the predetermined event time point, the processor loads an Effect asset and a prop from a resource database to output the modified content to the metaverse environment.

11. The apparatus of claim 8, wherein the user responsiveness information includes at least one type of information among facial expression information, gaze information, and motion information of the user, and
when the content is at a predetermined content start time point, the processor inputs the at least one type of information among the facial expression information, the gaze information, and the motion information of the user into the multimodal artificial intelligence model to acquire the user responsiveness.

12. The apparatus of claim 11, wherein the processor changes a scenario to correspond to a level of the acquired user responsiveness among scenarios of the content and output the changed scenario, or output and provide the changed scenario after a currently ongoing scenario.

13. The apparatus of claim 8, wherein the multimodal artificial intelligence model includes a plurality of convolutional neural networks, and each of the plurality of convolutional neural networks outputs a feature corresponding to each of facial expression information, gaze information, and motion information of the user.

14. The apparatus of claim 13, wherein the multimodal artificial intelligence model includes a recurrent neural network, and the recurrent neural network outputs a user responsiveness based on the facial expression information, the gaze information, and the motion information of the user which are output from each of the plurality of convolutional neural networks.

15. A training apparatus for providing content to a metaverse environment using an artificial intelligence, the training apparatus comprising:
a stimulus storage unit configured to store a stimulus moving image, a stimulus still image, or stimulus sound designed to induce an emotion related to a user responsiveness;
a reproducing unit configured to reproduce the content;
a photographing unit configured to acquire facial expression information, gaze information, posture information, and motion information of a user using at least one camera;
a user input processing unit configured to store specific sections set by the user; and
a labeling unit configured to provide content of the specific section to the user, acquire a responsiveness score based on a predetermined criterion, and label pieces of image sequence data of the specific section with the acquired responsiveness scores to generate training data.

16. The training apparatus of claim 15, wherein, when a preceding time point is set as an onset point and a following time point is set as an ending point among time points marked by the user clicking a mouse or a remote control, the specific section includes a part between the onset point and the ending point.
